(19) United States
(12) Patent Application Publication
(10) Pub. No.: US 2004/0210240 A1
(43) Pub. Date: Oct. 21, 2004

(54) Method and repair device for treating mitral valve insufficiency

(76) Inventor: Sean Saint, Santa Rosa, CA (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/418,834

(22) Filed: Apr. 21, 2003

(51) Int. Cl. A61B 17/10

(52) U.S. Cl. 606/139; 604/1

(57) ABSTRACT

A system, device and method for repairing mitral valve regurgitation is provided. A device is placed external to the mitral valve in the atrioventricular sulcus or groove of the heart and is cinched in to reduce the mitral valve annulus or the radius of curvature of the heart around the atrioventricular groove thus reducing the circumference of the mitral annulus.

### FIELD OF THE INVENTION

The present invention relates to a device and method for treating mitral annulus dilatation or mitral valve regurgitation.

### BACKGROUND OF THE INVENTION

The mitral valve of the heart is located between the left atrium and the left ventricle. In various types of cardiac disease, mitral valve insufficiency may result. Typically in cases where there is mitral valve insufficiency, there is some degree of annular dilatation and a condition of mitral valve regurgitation may thus result. Any one or more of the mitral valve structures, i.e., the anterior and posterior leaflets, the chordae, the papillary muscles or the annulus may be compromised by damage from disease or injury, causing mitral valve insufficiency. In some disease states, the left ventricle and correspondingly the mitral annulus become enlarged, causing mitral valve insufficiency. The ventricle enlarges and becomes spherical, pulling the papillary muscles and chordae away from the plane of the valve and further enlarging the regurgitant orifice. Mitral valve regurgitation occurs as the result of the leaflets being moved back from each other by the dilated annulus. The mitral valve insufficiency leads to disease progression and/or further enlargement and worsening of the insufficiency. Correction of the regurgitation may not require repair of the valve leaflets themselves, but simply a reduction in the size of the annulus and the sphericity of the left ventricle.

A variety of techniques have been attempted to reduce the diameter of the mitral annulus, improve coaptation of heart valve leaflets and eliminate or reduce valvular regurgitation in patients with incompetent valves. Current surgery to correct mitral regurgitation in humans includes number of mitral valve replacement and repair techniques. Valve replacement involves implanting a mechanical or biological valve. The valve replacement may result in a number of complications including a risk of endocarditis. Mechanical valve replacement requires subsequent anticoagulation treatment to prevent thromboembolisms.

As an alternative to valve replacement, various valve repair techniques have been used including quadrangular segmental resection of a diseased posterior leaflet; transposition of posterior leaflet chordae to the anterior leaflet; valvuloplasty with plication and direct suturing of the native valve; substitution, reattachment or shortening of chordae tendinae; and annuloplasty in which the effective size of the valve annulus is contracted by attaching a prosthetic annuloplasty ring to the endocardial surface of the heart around the valve annulus. The annuloplasty techniques may be used in conjunction with other repair techniques. Typically such rings are sutured along the posterior mitral leaflet adjacent to the mitral annulus in the left atrium. The rings either partially or completely encircle the valve, and may be rigid or flexible/non-elastic. All of these procedures require cardiopulmonary bypass, though some less and minimally invasive techniques for valve repair and replacement are being developed.

Another of such techniques involves diameter reduction or reduction in radius of curvature which includes placement of a circumferential mitral purse string suture in a periannular, subcoronary position (externally placed and mechanically reducing the circumference of the annulus). This, however, has resulted in a high surgical mortality rate in human patients with severe congestive heart failure. The procedure is also technically difficult.

Although mitral valve repair and replacement can successfully treat many patients with mitral valvular insufficiency, techniques currently in use are attended by significant morbity and mortality. Most valve repair and replacement procedures require a thoractomy, to gain access into the patient's thoracic cavity. Surgical intervention within the heart generally requires isolation of the heart and coronary blood vessels from the remainder of the arterial system and arrest of cardiac function. Open chest techniques with large sternum openings are typically used. These patients may have scarring retraction, tears or fusion of valve leaflets as well as disorders of the subvalvular apparatus.

Recently other surgical procedures have been provided to reduce the mitral annulus using a less invasive surgical technique. According to this method a prosthesis is transvenously advanced into the coronary sinus and the prosthesis is deployed within the coronary sinus to reduce the diameter of the mitral annulus. This may be accomplished in an open procedure or by percutaneously accessing the venous system by one of the internal jugular subclavion or femoral veins. The prosthesis is tightened down within the coronary sinus which is located adjacent the mitral annulus, to reduce the mitral annulus.

While the coronary sinus implant provides a less invasive treatment alternative, the placement of the prosthesis within the coronary sinus may be problematic for a number of reasons. Sometimes the coronary sinus is not accessible. The coronary sinus on a particular individual may not wrap around the heart far enough to allow enough encircling of the mitral valve. Also, leaving a device in the coronary sinus may result in formation and breaking off of thrombus which may pass into the right atrium, right ventrical and ultimately the lungs causing a pulmonary embolism. Another disadvantage is that the coronary sinus is typically used for placement of a pacing lead, which may be precluded with the placement of the prosthesis in the coronary sinus.

Accordingly, it would be desirable to provide a less invasive method and device for reducing an enlarged mitral annulus.

### SUMMARY OF THE INVENTION

The present invention provides a device and method for repairing mitral valve regurgitation. According to an embodiment of the invention, a device is placed external to the mitral valve in the atrioventricular sulcus or groove of the heart to reduce the mitral valve annulus or the radius of curvature of the heart around the atrioventricular groove and thus reduce the circumference of the mitral annulus.

According to one embodiment, the pericardial space adjacent the atrioventricular groove is accessed and the device is placed therein. A number of different techniques for accessing the pericardial space may be used including percutaneous, laparoscopic and open surgical techniques. In one embodiment, the device, once placed adjacent the groove, is cinched down to tighten the device around the atrioventricular groove, reducing the radius of curvature.

According to one embodiment the device is delivered percutaneously through a catheter that is located into the right atrium of the heart and then into the coronary sinus vessel. The device is then delivered out of the coronary sinus to reside in the atrioventricular groove.

According to another embodiment the device is delivered percutaneously through a catheter that is located into the right atrium of the heart and then into the pericardial space adjacent the coronary sinus vessel.

According to one embodiment of a delivery system of the invention, a system comprises a catheter for accessing the pericardial space, a mitral valve reducing device, and a mitral valve reducing device delivery member configured to place the reducing mechanism in the atrioventricular groove.

According to one embodiment, the catheter for accessing the pericardial space includes a device for accessing the pericardial space through the coronary sinus.

In one embodiment, the mitral valve reducing device comprises an elongated element that is naturally curved, and is introduced straight into the pericardial space, and thereafter released to return to its curved shape in which it reduces the radius of the mitral valve annulus. According to this embodiment, the device is created in several models, each having different lengths and curves. The physician may then select the appropriate one in view of the patient's anatomy.

In another embodiment, the mitral valve reducing device delivery system includes a cinching mechanism for cinching the mitral valve reducing mechanism to fit into the atrioventricular groove and reduce the mitral valve radius. A number of alternative ways of cinching a device are contemplated herein. For example, in one embodiment, the device is normally relatively straight and is caused to be formed into a reduced radius of curvature by a pull wire, tube or tether. The device may be made of a deformable elastic metal such as a Nitinol tube and the pull wire may be actuated to deform the Nitinol tube. In another embodiment, for example, the pull wire may plastically deform a yieldable metal (e.g. a tube constructed of stainless steel or martensite Nitinol or MP35N. The surface of the device may also have a textured or porous surface to promote tissue ingrowth. The device may also have a coating or infusion of a material or substance that promotes a tissue response that improves the device's gripping of the heart around the atrioventricular groove. The tissue may also be treated, e.g., by ablating or otherwise causing tissue adhesions or scarring around the device to improve device fixation within the groove.

### BRIEF DESCRIPTION OF THE DRAWINGS

FIG. 1A illustrates a diaphragmatic aspect of a heart.

FIG. 1B illustrates a sternocostal aspect of the heart of FIG. 1A.

FIG. 1C illustrates a top view of the heart of FIG. 1A in systole viewed from base with atria removed.

FIG. 1D illustrates a diaphragmatic aspect of the heart of FIG. 1A with an implanted mitral valve reducing device according to an embodiment of the invention, implanted in the atrioventricular groove.

FIG. 1E illustrates a sternocostal aspect of the heart of FIG. 1B with an implanted mitral valve reducing device according to an embodiment of the invention, implanted in the atrioventricular groove.

FIG. 1F illustrates a top view of the heart of FIG. 1C in systole viewed from base with atria removed, with a mitral valve reducing device according to an embodiment of the invention.

FIG. 2A illustrates a catheter placed in the coronary sinus of a heart to deliver the mitral valve reducing device according to an embodiment of the invention.

FIGS. 2B-2G illustrate the placement of a mitral valve reducing device through the coronary sinus into the atrioventricular groove according to one embodiment.

FIGS. 3A and 3B illustrate an enlarged view of the catheter used in FIGS. 2A-H.

FIG. 4A illustrates an embodiment of a mitral valve reducing device of the invention in a first position.

FIG. 4B illustrates the mitral valve reducing device of FIG. 4A in a second position.

FIG. 4C illustrates a cross section of the mitral valve reducing device of FIG. 4A along the lines 4C-4C.

FIG. 4D illustrates an enlarged cross section view of the device of FIG. 4A with a locking mechanism for locking the cinching wire in place.

FIG. 5A illustrates an embodiment of a mitral valve reducing device of the invention in a first position.

FIG. 5B illustrates the mitral valve reducing device of FIG. 5A in a second position.

FIG. 5C illustrates a cross section of the mitral valve reducing device of FIG. 5A along the lines 5C-5C.

FIG. 6A illustrates an embodiment of a mitral valve reducing device of the invention in a first position.

FIG. 6B illustrates the mitral valve reducing device of FIG. 6A.

FIG. 7A illustrates a mitral valve reducing device with an alternative cinching mechanism and locking mechanism of the invention.

FIG. 7B is a cross section of the device of FIG. 7A.

### DETAILED DESCRIPTION

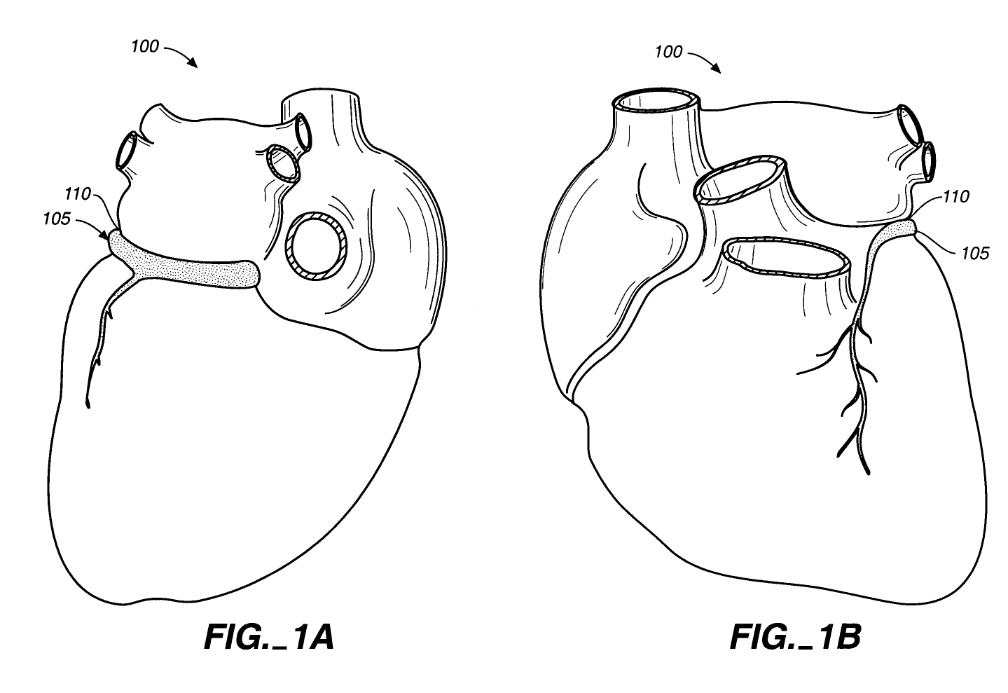

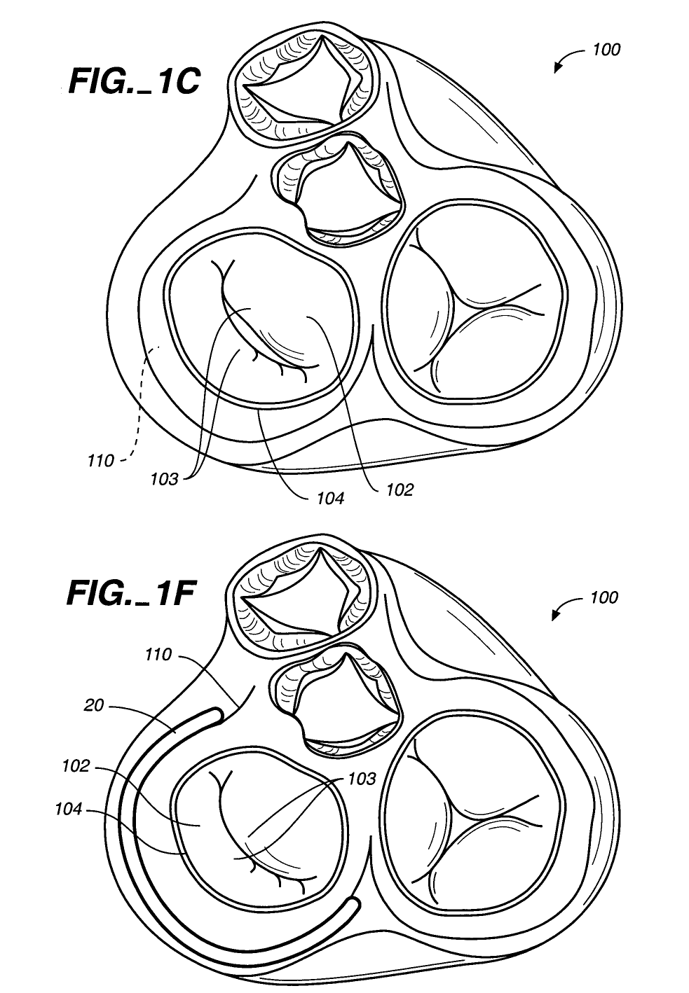

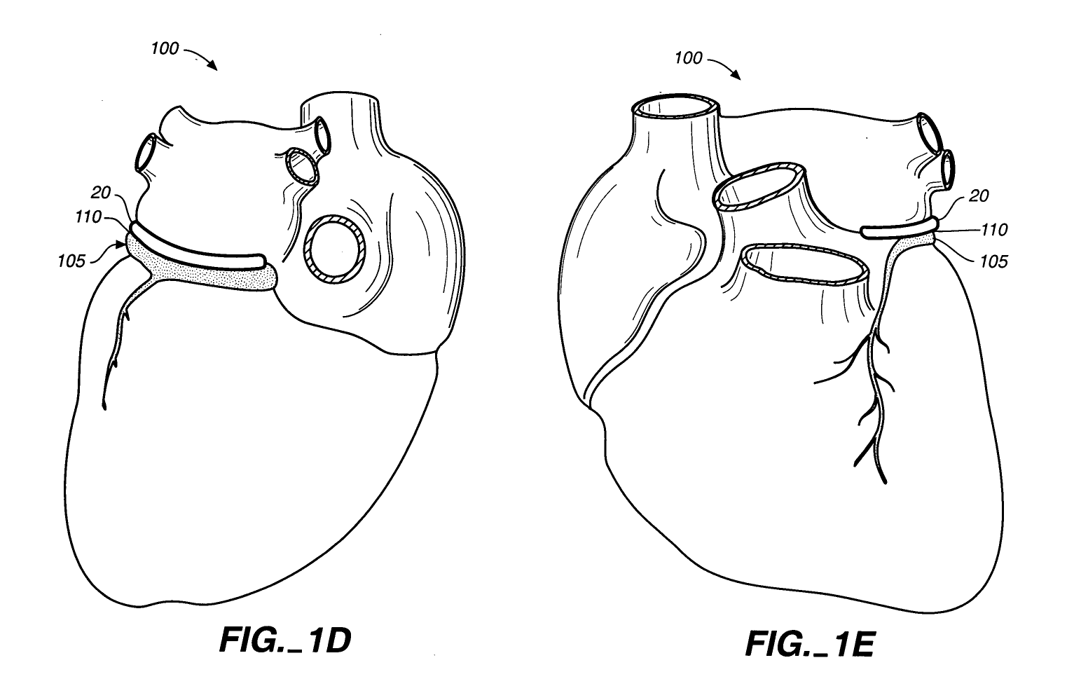

Referring to FIGS. 1A-1C, a heart 100 is illustrated prior to placement of a mitral valve reducing device 20. The coronary sinus 105 is located on the exterior of the heart 100, approximately around the atrioventricular sulcus or groove 110, which corresponds approximately to the mitral valve 102 within the heart 100. As illustrated in FIG. 1C, the leaflets 103 of the mitral valve 102 are moved back from each other when the heart is in systole, indicating mitral valve insufficiency.

Referring to FIGS. 1D-1F, a heart 100 is illustrated in which a mitral valve reducing device 20 is implanted. The device 20 is placed around the atrioventricular sulcus or groove 110 external of the heart muscle, external of the coronary sinus 105, and thus, approximately about the location of the mitral valve 102 or mitral annulus 104 of the heart. The device 20 operates to reduce circumference or radius of curvature of the mitral annulus 104 to bring the leaflets 103 of the valve 102 closer together when in systole.

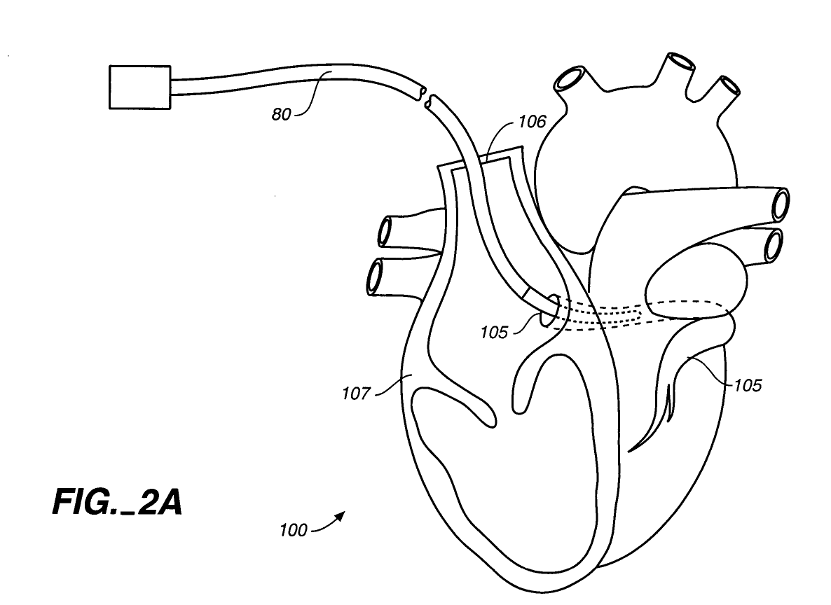

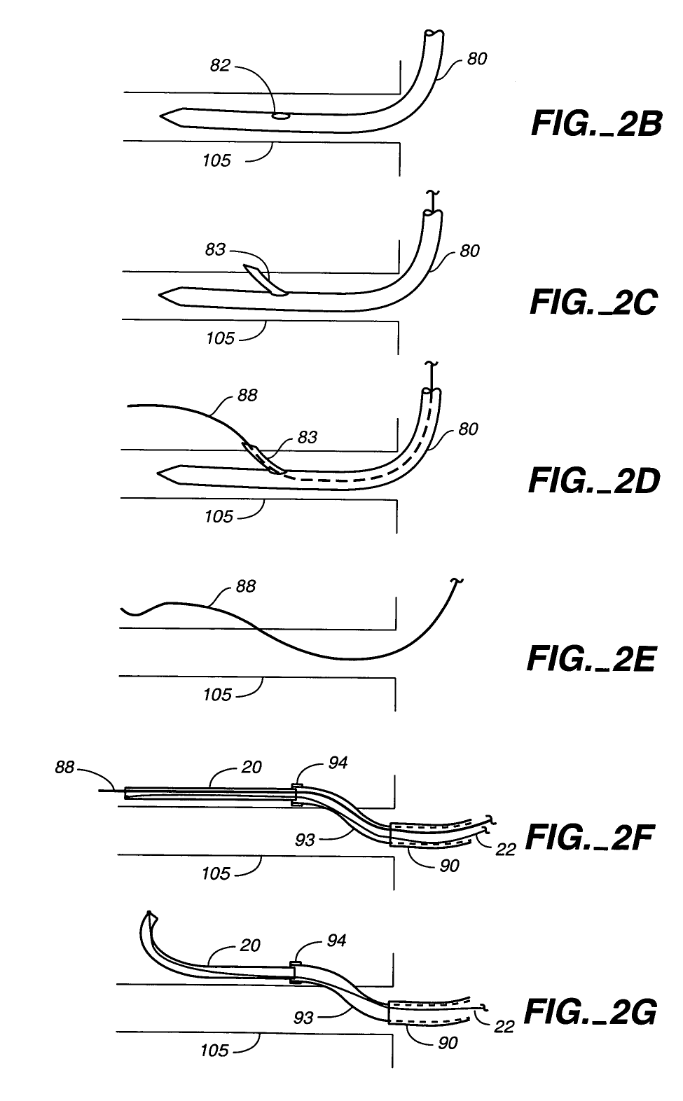

FIGS. 2A-2G illustrate an embodiment of a delivery system and method for placing the device 20 in the atrioventricular groove 110. As shown in FIG. 2A, a catheter 80 percutaneously accesses the vena cava 106 into the right atrium 107 where the coronary sinus 105 empties into the right atrium 107. The catheter 80 is directed through the coronary sinus 105 in order to access the atrioventricular groove 110 adjacent the coronary sinus 105 (FIGS. 2A and 2B).

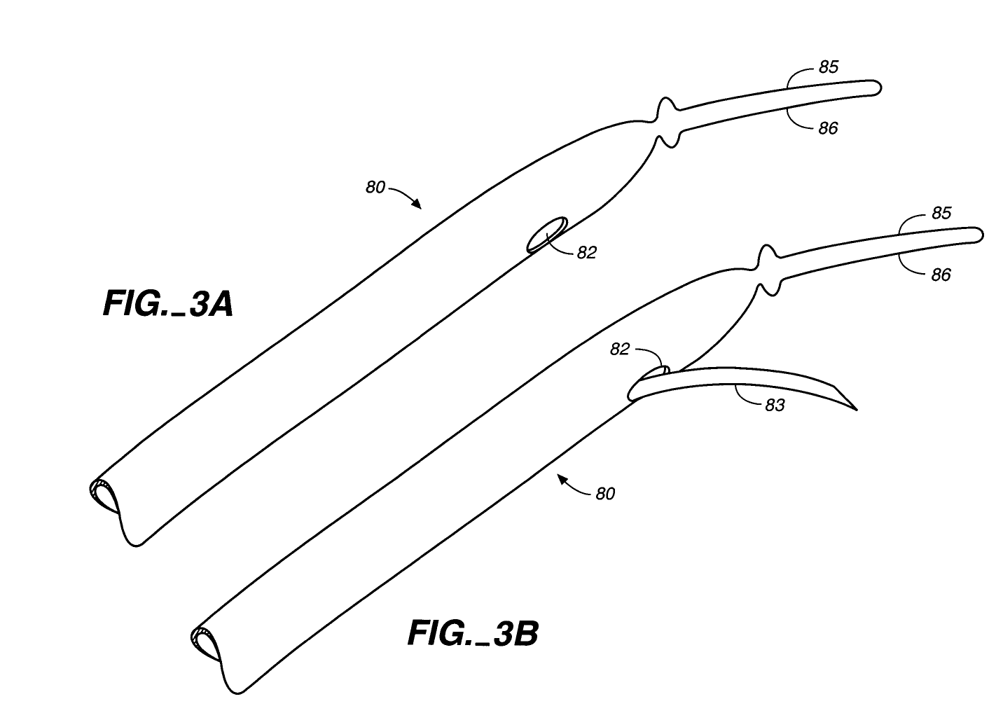

A catheter 80 that may be used to access the atrioventricular groove 110 through the coronary sinus 105 is shown in FIGS. 3A and 3B. The catheter 80 may be constructed in a manner similar to the Cross point TransAccess™ catheter of TVI, Inc. where the catheter tip 85 includes an imaging device 86 that allows visualization of the catheter 80 as it is placed through the coronary sinus 105. The catheter 80 also includes a side opening 82 that guides a hollow needle 83 through a side of a vessel in which the catheter 80 is located. The hollow needle 83 may be retracted into the catheter as illustrated in FIG. 3A while the catheter 80 is positioned. The hollow needle 83 may then be extended from the opening 82 at an angle with respect to the catheter tip 85, to puncture an opening in a vessel containing the catheter 80.

As illustrated in FIG. 2C, a side opening 82 in the catheter 80 guides the hollow needle 83 to puncture the coronary sinus 105 to access the space adjacent the atrioventricular groove 110 (FIG. 2A). As illustrated in FIG. 2D, a guide wire 88 is guided through the needle 83 into position adjacent the atrioventricular groove 110 (FIG. 2A). The needle 83 is removed into the catheter 80 and the catheter 80 is removed as illustrated in FIG. 2E, leaving the guide wire 88 in place.

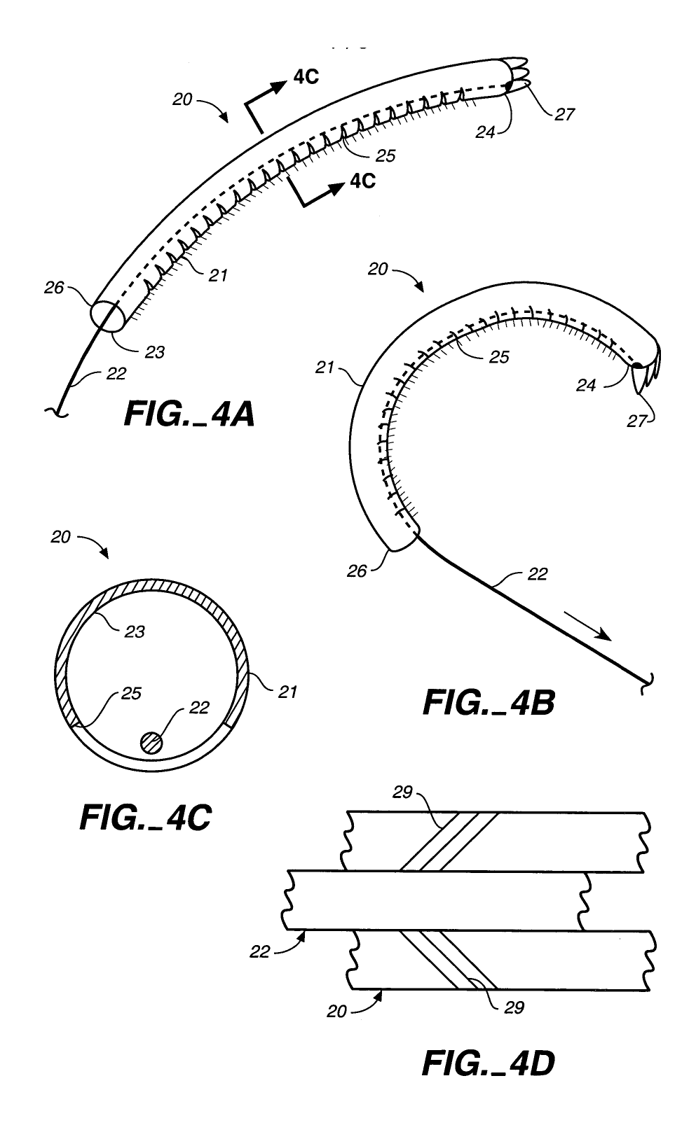

As illustrated in FIG. 2F, a delivery catheter 90 is introduced into the coronary sinus 105. A device 20 is then delivered through a delivery catheter 90 over the guidewire 88 through and out of the coronary sinus 105 adjacent the atrioventricular groove 110 (FIG. 2A) using a push rod 93 that is coupled to the proximal end 26 of the device 20 with a releasable locking mechanism 94. As illustrated in FIG. 2G, the guidewire 88 is then removed. A wire 22 (or other tether or tube) bonded to the distal end 24 of the device 20 is then used to cinch the device 20, reducing its radius of curvature and fixing the device within the groove 110. (FIGS. 1D-1F). The device 20 is locked into its cinched position with a cinch locking mechanism, for example, as described below with respect to FIG. 4D and FIGS. 7A-7B. The releasable locking mechanism 94 may then be actuated to release the push rod 93 from the device 20. As an alternative to delivering the device 20 through catheter 90, the push rod 93 and device 20 may be introduced over the guidewire 80 separately. After the device has been deployed, a covered stent may be place in the coronary sinus to repair the opening through he artery.

Alternative means of accessing and navigating the pericardial space may be used such as, for example as described in U.S. Pat. Nos. 6,162,195 and 5,827,216 incorporated herein by reference. The space adjacent the coronary sinus may also be accessed directly from the right atrium rather than through the coronary sinus, for example using a needle or catheter with imaging capabilities and following the coronary sinus to the location of the atrioventricular groove. Alternatively, the pericardial space may be accessed in an open surgical procedure.

FIGS. 4A -4D illustrate one embodiment of a device 20 that may be delivered and deployed as illustrated in FIGS. 1A-F and 2A-G. The device 20 comprises an elongate member 21 configured to be delivered in a first extended position as illustrated in FIG. 4A and to be cinched into a curved configuration as shown in FIG. 4B. The elongate member 21 includes a wire 22 coupled on the distal end 24 of the device 20 and extending through a hollow lumen 23 through the elongate member 21 out of a proximal end 26 of the elongate member 21. The wire 22 is of sufficient length to extend through the push tube 93, through the catheter 90 (FIG. 2F), and out of the proximal end of the catheter 90. The elongate member 21 includes cut outs 25 on one long side of the elongate member 21 that permit bending of the device 20 in one direction when the wire 22 is pulled while the device 20 is held in place by the push tube 93. The elongate member 21 includes a locking mechanism 28 comprising a plurality of barbs 29 within the lumen 23 oriented in one direction to that the wire 22 may be pulled in a direction to cinch the device 20 while preventing the wire from moving in the opposite direction in which the device 20 will straighten. The elongate member 21 further comprise a plurality of fingers members 26 affixed to the distal end 24 of the device 20. The finger members 26 act to engage the heart to provide greater adherence and/or gripping to the heart tissue when the device 20 is deployed. The finger members may be constructed of an elastic metal such as martensitic Nitinol and are attached to or integral with to the distal end of the device 20. The device 20 is formed of an elastic metal such as martensitic Nitinol or may be formed of a material such as a metal that plastically deforms when cinching the device into a reduced diameter and that retains reduced diameter after it is deployed. The device 20 also comprises surface features 28 for gripping the heart when the device 20 is deployed. The surface features may include for example, structures or shapes that increase the surface area of the device 20 at least in part where the device 20 is intended to grip the heart. Alternatively or additionally, at least a portion of the device's surface may include a porous surface (open or closed pore) to promote tissue ingrowth, or a coating or infused material or substance that promotes ingrowth, tissue adhesion or gripping of the heart by the device.

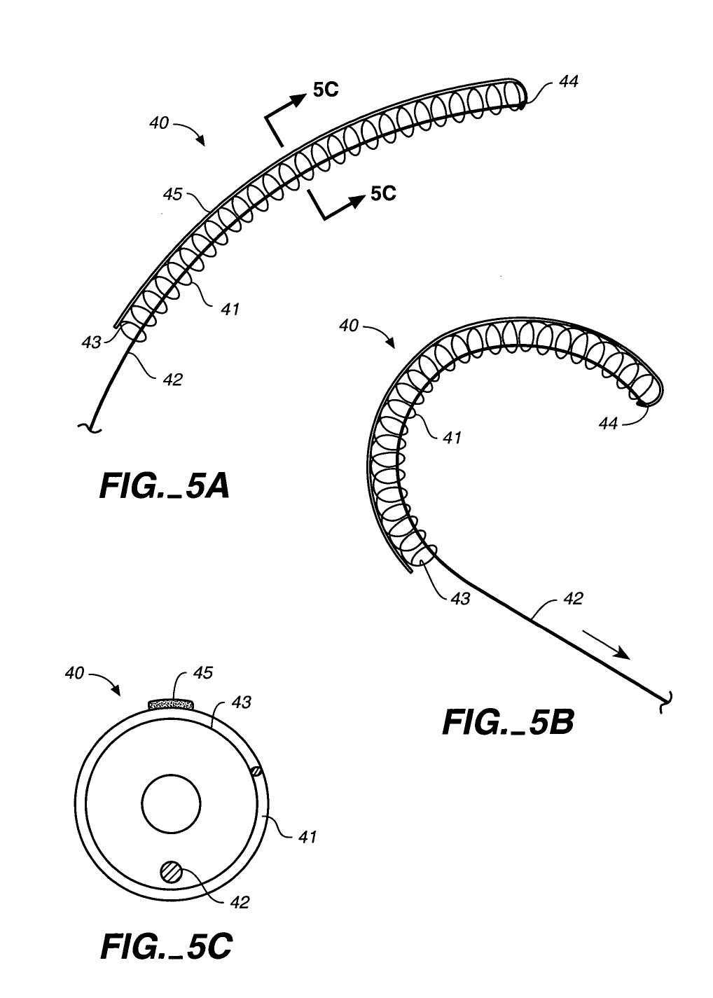

FIGS. 5A and 5B illustrate another embodiment of a device 40 that may be delivered and positioned in a manner similar to the device 20 as described above with respect to FIGS. 1A-F and 2A-H. The device 40 comprises a wire coil 41 having a cinching wire 42 bonded on one end 44 of the device 40 and extending through a lumen 43 formed by the coil 41. The coil 41 has a glue 45 or other material along one the length on one side of the coil 41 so that when the actuating wire 42 extending through the lumen 43 is pulled to bend the coil 41, the coil 41 bends at a preferred, unglued side. In use, the device 40 is placed though a delivery catheter and over a guidewire and positioned adjacent the atrioventricular groove 110 in a similar manner as device 20 is delivered and positioned as described herein. The coil 41 is bent by actuating or pulling the wire 42 while stabilizing the device 40 with a tool preferably placed through the delivery catheter. The device 40 may be formed of an elastic metal such as martensitic Nitinol (in which case a cinching locking mechanism is used such as that described above with respect to the device 40) or may be formed of a material such as a metal that plastically deforms when cinching the device into a reduced diameter and that retains reduced diameter after it is deployed.

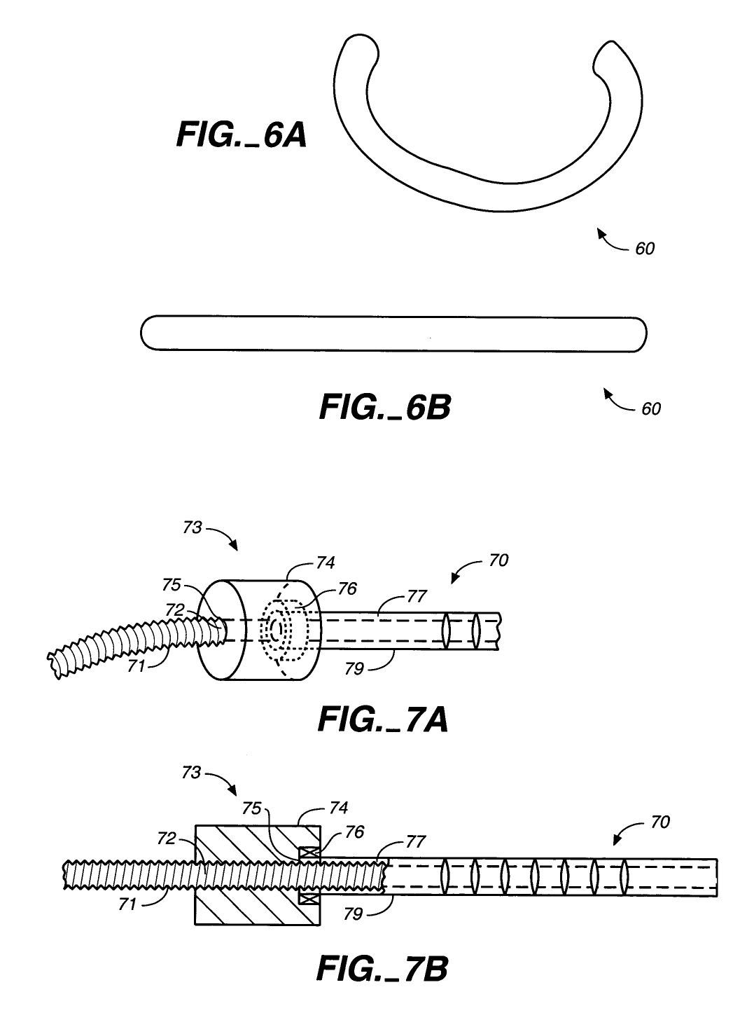

Referring to FIGS. 6A and 6B, another embodiment of the mitral valve reducing device of the invention is illustrated. As illustrated in FIG. 6A, the device 60 is illustrated in its first and naturally curved shape. The device 60 is introduced through a catheter in which it is held in a straight position as illustrated in FIG. 6B. When released into the atrioventricular groove, the device 60 tends to return to its natural shape as illustrated in FIG. 6A. The device 60 may be provided in several different lengths and curvatures so that a particular size may be selected from a plurality of different sizes and shapes depending upon the patient's anatomy. The device 60 is delivered in a manner similar to that shown in FIGS. 1A-F and FIGS. 2B-2E. A delivery catheter similar to catheter 90 is the placed over a guidewire to the location at the atrioventricular groove where the device 60 is to be released. The device 60 is released from the catheter whereupon it returns to a curved position within the atrioventricular groove to reduce the radius of curvature of the mitral annulus.

Referring to FIGS. 7A and 7B, an alternative mitral valve reducing device 70 is illustrated comprising an elongate member 79 that is constructed in a manner similar to the device 20 described above. A pull wire or pull tube 71 comprises a threaded distal end 72 extending through a bearing screw element 73 that comprises a cylinder 74 and a threaded inner lumen 75 that receives the threaded distal end of the pull tube 71. The bearing screw element 73 further comprises a bearing connecting element 76 that connects to the proximal end 77 of the pull tube 71 so that the cylinder 74 of the bearing screw element 73 may be rotated without rotating the mitral valve reducing device 70. In use, the device 70 is delivered in a straight position. The device is then cinched into a curved position by rotating the cylinder 74 in a direction in which the draws the threaded tube 71 through the threaded lumen 75 of the cylinder 74 in a proximal direction. The device 70 may be straightened if, for example, it is not properly placed, by rotating the cylinder 74 in the opposite direction in which the threaded tube 71 moves in distal direction. The device 70 may then be repositioned and replaced.

In one embodiment the device 20, 40, 60 or 70 is coated or infused with a material, substance or agent that promotes fibrosing, tissue ingrowth or growth of tissue around the device. For example, the device may be at least partially coated or infused with a substance that promotes healing or tissue ingrowth, fro example, fibrinogen or plasma treated in absence of ammonia or collagen. Also, the device may comprise a material that has inherent porosity such as polypropylene, polyurethane, latex or other suitable material, or combinations of materials, which renders at least a portion of the surface of the device, suitable for ingrowth of tissue or matter. As used herein, “porous” means that openings are formed on at least the surface of the material facing outwardly toward the interior of the vessel. As such, “porous” may include materials which have dimples or depressions positioned on the surface thereof, closed-cell pores which extend partially through the thickness of the material, open-cell pores which form a channel through the thickness of the material, and both regularly and irregularly-shaped and sized pores. The device may thus be formed from material having the desired porosity to enhance ingrowth, or the device may be formed from a material lacking the desired porosity which is then coated or treated with a material providing the surface with the desired porosity (e.g., metal coated with latex).

In another embodiment, an inflammatory response acting agent for example collagen, is coated on (or infused in) at least a portion of the mitral valve reducing device to cause an inflammatory response or scar tissue to form around the valve, the body's response causing the device to be sealed down to have a better grip on the heart. In one embodiment the coated device is placed adjacent the aterioventricular groove and then after a period of time in which tissue growth or an inflammatory response occurs around the device, the device is again surgically accessed and is cinched down around the valve. In an alternative embodiment, an RF ablation catheter such as one that is known in the art is used to ablate the tissue adjacent the mitral valve reducing device to cause scar tissue to form around the device to provide adherence of the heart or connective tissue to the mitral valve reducing device.

While the invention has been described with reference to particular embodiments, it will be understood to one skilled in the art that variations and modifications may be made in form and detail without departing from the spirit and scope of the invention.

What is claimed is:

1. A method of reducing the circumference of a mitral valve annulus comprising the steps of:
providing a mitral valve reducing device comprising an elongate member;
positioning the elongate member in a first position at a location adjacent the atrioventricular groove and exterior of the vasculature of the heart, wherein in the first position, the elongate member has a first radius of curvature; and
releasing the elongate member in the atrioventricular groove in a second position wherein in the second position the elongate member has a second radius of curvature less than the first radius of curvature so as to reduce the circumference of the mitral valve annulus.

2. The method of claim 1 wherein the step of positioning the elongate member in a first position comprises delivering the elongate member along a coronary sinus to the location adjacent the atrioventricular groove.

3. The method of claim 1 wherein the step of positioning the elongate member in a first position comprises delivering the elongate member through a coronary sinus vessel, forming an opening in the coronary sinus and delivering the elongate member out of the coronary sinus through the opening.

4. The method of claim 1 wherein the step of positioning the elongate member in a first position comprises accessing and navigating the pericardial space.

5. The method of claim 1 wherein the step of releasing the elongate member in the atrioventricular groove in a second position comprises bending the elongate member from the first position into the second position.

6. The method of claim 5 wherein the step of providing an elongate member comprises providing an elongate member comprising a cinching mechanism; and wherein the step of bending the elongate member comprises actuating the cinching mechanism.

7. The method of claim 6 wherein the step of providing an elongate member comprises providing an elongate member having a preferential bending mechanism at a location along a length of the elongate member; and
wherein the step of bending the elongate member comprises bending the elongate member at the location.

8. The method of claim 1 further comprising the step of increasing tissue adherence to the elongate member.

9. The method of claim 8 wherein the step of increasing tissue adherence occurs prior to releasing the elongate member in the atrioventricular groove in the second position.

10. The method of claim 8 wherein the step of increasing the tissue adherence comprises ablating adjacent tissue.

11. The method of claim 8 wherein the step of increasing the tissue adherence comprises:
providing a coating on the elongate member to cause a tissue response.

12. The method of claim 11 wherein the step of providing a coating comprises providing a coating comprising a tissue growth promoter to cause tissue growth around the elongate member.

13. The method of claim 11 wherein the step of providing a coating comprises providing a coating comprising an inflammatory response agent.

14. A system for reducing the mitral annulus of a heart comprising:
a mitral valve reducing device comprising:
an elongate member having a first position wherein in the first position, the elongate member has a first radius of curvature, and a second position wherein in the second position the elongate member has a second radius of curvature less than the first radius of curvature; and
a delivery system comprising:
a catheter configured to access a pericardial space of a heart, adjacent an atrioventricular groove and outside of vasculature of the heart; and
a mitral valve reducing device delivery member configured to place the reducing mechanism in the atrioventricular groove.

15. The system of claim 14 wherein the catheter comprises a puncture needle configured to puncture a vessel in which the catheter resides to access the pericardial space.

16. The system of claim 14 wherein the mitral valve reduction device comprises a cinching mechanism; and wherein the step of bending the elongate member comprises actuating the cinching mechanism.

17. The system of claim 14 wherein the mitral valve reduction device comprises a preferential bending mechanism at a location along a length of the elongate member.

18. A mitral valve reduction device comprising:
an elongate member having a first position wherein in the first position, the elongate member has a first radius of curvature, a second position wherein in the second position the elongate member has a second radius of curvature less than the first radius of curvature, and a bending mechanism configured to move the elongate member from the first position to the second position; and
a coating on the elongate member, wherein the coating comprises a tissue response promoter for promoting a tissue response adjacent the elongate member.

19. The mitral valve reduction device of claim 18 wherein the coating comprises a tissue growth promoter.

20. The mitral valve reduction device of claim 18 wherein the coating comprises a tissue inflammation promoter.

21. A mitral valve reduction device comprising:
an elongate member having a first position wherein in the first position, the elongate member has a first radius of curvature and a second position wherein in the second position the elongate member has a second radius of curvature less than the first radius of curvature;
an end comprising a tissue engaging element configured to engage the heart within the atrioventricular groove.

22. The mitral valve reduction device of claim 21 wherein the tissue engaging element comprises at least one finger member extending from the end of the elongate member.

23. The mitral valve reduction device of claim 21 further comprising:
a bending mechanism configured to move the elongate member from the first position to the second position.
